(19) United States
(12) Patent Application Publication
(10) Pub. No.: US 2016/0024521 A1
(43) Pub. Date: Jan. 28, 2016

(54) RETROGRADE DELIVERY OF SDF-1 FOR TREATMENT OF MYOCARDIAL INFARCTION

(71) Applicants: THE CLEVELAND CLINIC FOUNDATION, Cleveland,, OH (US); JUVENTAS THERAPEUTICS, INC., Cleveland,, OH (US)

(72) Inventors: Marc S. PENN, Beachwood, OH (US); Rahul ARAS, Broadview Hight, OH (US); Joseph PASTORE, Mentor, OH (US); Scott J. FISHER, Solon, OH (US)

(21) Appl. No.: 14/773,984

(22) PCT Filed: Mar. 15, 2014
(86) PCT No.: PCT/US14/29921

§ 371 Date: Sep. 9, 2015

Related U.S. Application Data
(60) Provisional application No. 61/792,954, filed on Mar. 15, 2013.

(51) Int. Cl. C12N 15/85; A61M 25/10

(52) U.S. Cl. CPC C12N 15/85; A61M 25/10; A61M 2025/105; C12N 2800/107

(57) ABSTRACT

Described herein are methods of treating a subject with a cardiomyopathy by administering to the subject's heart, via percutaneous retrograde coronary sinus perfusion, a pharmaceutical composition that comprises a DNA plasmid encoding SDF-1 and a pharmaceutically acceptable carrier or diluent.

### CROSS-REFERENCE TO RELATED APPLICATIONS

This application claims the benefit of U.S. Provisional Patent Application 61/792,954, filed Mar. 15, 2013, which is incorporated herein by reference in its entirety.

### TECHNICAL FIELD

The inventions disclosed herein relate to the field of treatments for ischemic heart disease.

### BACKGROUND

Heart failure (HF) is one of the leading causes of morbidity and mortality in Westernized countries. Even in the presence of current guideline-based therapy, HF has a US prevalence of 6.6 M Americans and an incidence of 670,000 new cases/year. HF is extremely costly to the health care system. The estimated direct and indirect cost of chronic HF in the United States for 2008 is $34.8 billion, largely due to the recurrent, lengthy hospitalizations associated with the disease. Novel effective HF treatments will improve quality of life and reduce the number of HF hospitalizations which will provide both clinical benefit and savings to the health care system.

One of the primary causes of HF is previous damage due to ischemic cardiovascular disease including myocardial infarction (MI). A majority of ischemic HF patients have systolic dysfunction, or impaired cardiac pumping ability. Based on recent clinical trial data, it is estimated that 62-67% of patients with systolic HF have HF caused by previous ischemic heart disease. This segment of the HF population has a large potential to benefit from therapeutic intervention.

Recently, both HF of ischemic etiology and its precursor, acute MI, have been targeted for treatment by stem cell-based regenerative medicine with promising initial results. Regenerative medicine has a high therapeutic potential for treatment of ischemic cardiac disease because, unlike current treatments, which focus on either alleviating symptoms or reducing cardiac work load, regenerative medicine provides an opportunity to repair and retain function in degenerating organs. Preliminary efficacy (i.e. improvement in cardiac function) has been shown in a number of clinical trials. While clinically promising, stem cell therapies face challenges related to the complicated and time-consuming process of obtaining and preparing the cells for clinical use.

### SUMMARY

Described herein are methods of treating a subject with an ischemic by comprising administering to the subject's heart, via percutaneous retrograde coronary sinus perfusion, a pharmaceutical composition that comprises a DNA plasmid encoding SDF-1 and a pharmaceutically acceptable carrier or diluent.

### BRIEF DESCRIPTION OF THE DRAWINGS

FIG. 1 shows luciferase expression following retrograde infusion of plasmid encoding luciferase. Expression measured by bioluminescence imaging from excised pig hearts dosed with 5 mg (A and B) or 15 mg (C and D) of plasmid.

FIG. 2 shows luciferase expression following retrograde infusion of plasmid encoding luciferase. Colored regions represent areas of protein expression. Anterior (A and D), posterior (B and E) and intramyocardial (C and F) perspectives are shown for two separate hearts.

FIG. 3 shows changes in LVEF (A), LVESV (B) and wall motion score index (WMSI) (C) at 60 days post-dose of retrograde coronary sinus deliver of JVS-100 in a porcine model of heart failure.

FIG. 4 depicts a fluoroscopic image of a balloon catheter in the coronary sinus for retrograde infusion.

### DETAILED DESCRIPTION OF ILLUSTRATIVE EMBODIMENTS

Unless defined otherwise, all technical and scientific terms used herein have the same meaning as is commonly understood by one of skill in the art to which the application(s) belong. All patents, patent applications, published applications and publications, Genbank sequences, websites and other published materials referred to throughout the entire disclosure herein, unless noted otherwise, are incorporated by reference in their entirety. In the event that there are a plurality of definitions for terms herein, those in this section prevail. Unless otherwise defined, all technical terms used herein have the same meaning as commonly understood by one of ordinary skill in the art to which this application belongs. Commonly understood definitions of molecular biology terms can be found in, for example, Rieger et al., Glossary of Genetics: Classical and Molecular, 5th edition, Springer-Verlag: New York, 1991; and Lewin, Genes V, Oxford University Press: New York, 1994.

Methods involving conventional molecular biology techniques are described herein. Such techniques are generally known in the art and are described in detail in methodology treatises, such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981. Chemical synthesis of nucleic acids can be performed, for example, on commercial automated oligonucleotide synthesizers. Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992. Conventional methods of gene transfer and gene therapy can also be adapted for use in the application. See, e.g., Gene Therapy: Principles and Applications, ed. T. Blackenstein, Springer Verlag, 1999; Gene Therapy Protocols (Methods in Molecular Medicine), ed. P. D. Robbins, Humana Press, 1997; and Retro-vectors for Human Gene Therapy, ed. C. P. Hodgson, Springer Verlag, 1996.

Where reference is made to a URL or other such identifier or address, it understood that such identifiers can change and particular information on the internet can come and go, but equivalent information can be found by searching the internet. Reference thereto evidences the availability and public dissemination of such information.

As used herein, “nucleic acid” refers to a polynucleotide containing at least two covalently linked nucleotide or nucleotide analog subunits. A nucleic acid can be a deoxyribonucleic acid (DNA), a ribonucleic acid (RNA), or an analog of DNA or RNA. Nucleotide analogs are commercially available and methods of preparing polynucleotides containing such nucleotide analogs are known. The nucleic acid can be single-stranded, double-stranded, or a mixture thereof. For purposes herein, unless specified otherwise, the nucleic acid is double-stranded, or it is apparent from the context.

As used herein, “DNA” is meant to include all types and sizes of DNA molecules including cDNA, plasmids and DNA including modified nucleotides and nucleotide analogs.

As used herein, “nucleotides” include nucleoside mono-, di-, and triphosphates. Nucleotides also include modified nucleotides, such as, but are not limited to, phosphorothioate nucleotides and deazapurine nucleotides and other nucleotide analogs.

As used herein, the term “subject” refers to animals such as mammals and birds, including humans, primates, rodents, cattle, pigs, rabbits, goats, sheep, mice, rats, guinea pigs, cats, dogs, horses, chicken and others.

As used herein, “administering” to a subject is a procedure by which one or more delivery agents and/or large nucleic acid molecules, together or separately, are introduced into or applied onto a subject such that target cells which are present in the subject are eventually contacted with the agent and/or the large nucleic acid molecules.

As used herein, “expression” refers to the process by which nucleic acid is translated into peptides or is transcribed into RNA, which, for example, can be translated into peptides, polypeptides or proteins. If the nucleic acid is derived from genomic DNA, expression may, if an appropriate eukaryotic host cell or organism is selected, include splicing of the mRNA. For heterologous nucleic acid to be expressed in a host cell, it must initially be delivered into the cell and then, once in the cell, ultimately reside in the nucleus.

As used herein the term “cardiomyopathy” refers to the deterioration of the function of the myocardium (i.e., the actual heart muscle) for any reason. Subjects with cardiomyopathy are often at risk of arrhythmia, sudden cardiac death, or hospitalization or death due to heart failure.

As used herein, the term “ischemic cardiomyopathy” is a weakness in the muscle of the heart due to inadequate oxygen delivery to the myocardium with coronary artery disease being the most common cause.

As used herein the term “ischemic cardiac disease” refers to any condition in which heart muscle is damaged or works inefficiently because of an absence or relative deficiency of its blood supply; most often caused by atherosclerosis, it includes angina pectoris, acute myocardial infarction, chronic ischemic heart disease, and sudden death.

As used herein the term “myocardial infarction” refers to the damaging or death of an area of the heart muscle (myocardium) resulting from a blocked blood supply to that area.

As used herein the term “6-minute walk test” or “6MWT” refers to a test that measures the distance that a patient can quickly walk on a flat, hard surface in a period of 6 minutes (the 6MWD). It evaluates the global and integrated responses of all the systems involved during exercise, including the pulmonary and cardiovascular systems, systemic circulation, peripheral circulation, blood, neuromuscular units, and muscle metabolism. It does not provide specific information on the function of each of the different organs and systems involved in exercise or the mechanism of exercise limitation, as is possible with maximal cardiopulmonary exercise testing. The self-paced 6MWT assesses the submaximal level of functional capacity. (See for example, AM J Respir Crit Care Med, Vol. 166. Pp 111-117 (2002)).

As used herein “New York Heart Association (NYHA) functional classification” refers to a classification for the extent of heart failure. It places patients in one of four categories based on how much they are limited during physical activity; the limitations/symptoms are in regards to normal breathing and varying degrees in shortness of breath and or angina pain:

| NYHA | |
|---|---|
| Class | Symptoms |
| I | No symptoms and no limitation in ordinary physical activity, |
| | e.g. shortness of breath when walking, climbing stairs etc. |
| II | Mild symptoms (mild shortness of breath and/or angina) and |
| | slight limitation during ordinary activity. |
| III | Marked limitation in activity due to symptoms, even during |
| | less-than-ordinary activity, e.g. walking short distances |
| | (20-100 m). Comfortable only at rest. |
| IV | Severe limitations. Experiences symptoms even while at rest. |
| | Mostly bedbound patients. |

This application relates to compositions and methods for treating a cardiomyopathy in a subject that results in reduced and/or impaired myocardial function. The cardiomyopathy treated by the compositions and methods herein can include cardiomyopathies associated with a pulmonary embolus, a venous thrombosis, a myocardial infarction, a transient ischemic attack, a peripheral vascular disorder, atherosclerosis, ischemic cardiac disease and/or other myocardial injury or vascular disease.

In certain embodiments of the invention, the patient is one exhibiting HF of ischemic etiology and may have a known history of systolic dysfunction and/or prior MI. Patients may have a well-defined area of regional dysfunction defined as 3 consecutive abnormal wall motion segments on echocardiography. Symptomatic systolic heart failure patients exhibit reduced 6 minute walk distance, have enlarged hearts, reduced ability to perform exercise, and poor quality of life compared to healthy patients. They may also exhibit elevated NTproBNP concentrations.

Percutaneous retrograde coronary sinus perfusion is a well-established alternative route of administration that has been shown to be safe and feasible for the delivery of biologics in preclinical models and clinical trials. The methodology associated with this technique generally involves, using aseptic technique and local anesthesia, inserting a guide sheath into the internal jugular vein, subclavian vein, antecubital vein, brachial vein, the femoral vein, a radial vein, or other suitable entry point, followed by advancing a balloon catheter guided by a wire using standard procedures into the vein. Once in the inferior vena cava, the catheter is advanced into the right atrium and then rotated along the posterior atrial wall to a site just above the septal leaflet or the tricuspid valve. Following gentle advancement of the catheter into the coronary sinus, the balloon is placed in a non-obstructing mid-position. The balloon should be positioned in the coronary sinus in one of the following positions near the infarcted area as determined by the treating physician's clinical judgment: 1) the coronary sinus; 2) the middle cardiac vein; 30 the lesser (small) cardiac vein; or 4) the great cardiac vein. The particular characteristics of each case may require placement of the balloon in a position other than these four positions, however. Once in place, the balloon is inflated (preferably to a pressure of no more than about 2 ATM) and the medicament to be perfused is infused through the catheter lumen into the coronary sinus over a specified period of time (preferably, about 2 minutes). In some embodiments the balloon will remain inflated for about 10 minutes after infusion to permit the retrograde diffusion of the medicament into the cardiac tissue.

Described herein are methods of treating a subject with a cardiomyopathy by administering to the subject's heart, via percutaneous retrograde coronary sinus perfusion, a polynucleotide encoding stromal cell-derived factor-1 (SDF-1). In some embodiments of the described method polynucleotide encoding SDF-1 is a DNA plasmid. In some embodiments of the described method the DNA plasmid encoding SDF-1 includes the sequence of SEQ ID NO: 1.

Described herein are methods of treating a subject with a non-ischemic cardiomyopathy by administering to the subject's heart, via percutaneous retrograde coronary sinus perfusion, a polynucleotide encoding stromal cell-derived factor-1 (SDF-1). In some embodiments of the described method polynucleotide encoding SDF-1 is a DNA plasmid. In some embodiments of the described method the DNA plasmid encoding SDF-1 includes the sequence of SEQ ID NO: 1.

Described herein are methods of treating a subject with an ischemic cardiomyopathy by administering to the subject's heart, via percutaneous retrograde coronary sinus perfusion, a polynucleotide encoding stromal cell-derived factor-1 (SDF-1). In some embodiments of the described method polynucleotide encoding SDF-1 is a DNA plasmid. In some embodiments of the described method the DNA plasmid encoding SDF-1 includes the sequence of SEQ ID NO: 1.

The methods described herein may be used to treat a subject suffering from an acute myocardial infarction. The described methods may also be used to treat a subject with a known history of chronic systolic dysfunction. In addition, these methods may be employed to treat a subject who has previously suffered a myocardial infarction. In some embodiments the describe method may include identifying a subject that is having or has had a myocardial infarction, or that has a history chronic systolic dysfunction, and administering to the subject's heart a polynucleotide encoding SDF-1 via retrograde coronary sinus perfusion.

Described herein are compositions comprising at least one of the described polynucleotides encoding SDF-1 and a pharmaceutically acceptable carrier. The described compositions are useful, for example, for administration to a subject to treat cardiomyopathy. Such compositions are useful, for example, for administration to a subject to treat ischemic cardiomyopathy, such as those described and exemplified herein. The described compositions are useful, for example, for administration to a subject to treat non-ischemic cardiomyopathy. The compositions may be formulated as any of various preparations that are known and suitable in the art, including those described and exemplified herein. In some embodiments, the compositions are aqueous formulations. Aqueous solutions may be prepared by admixing the antibodies or antigen-binding fragments in water or suitable physiologic buffer, and optionally adding suitable colorants, flavors, preservatives, stabilizing and thickening agents and the like as desired. Aqueous suspensions may also be made by dispersing the polynucleotide in water or physiologic buffer with or without a viscous material, such as natural or synthetic gums, resins, methylcellulose, sodium carboxymethylcellulose, and other well-known suspending agents. In some embodiments of the described method includes a the use of a composition including a DNA plasmid encoding SDF-1 and a pharmaceutically acceptable carrier. In some embodiments of the described method includes the use of a composition including a DNA plasmid encoding SDF-1 having the sequence of SEQ ID NO: 1 and a pharmaceutically acceptable carrier.

As described herein, the pharmaceutically acceptable carrier may be a solution having a certain percentage of dextrose. In some embodiments the pharmaceutically acceptable carrier contains about 1% dextrose. In some embodiments the pharmaceutically acceptable carrier contains about 2% dextrose. In some embodiments the pharmaceutically acceptable carrier contains about 3% dextrose. In some embodiments the pharmaceutically acceptable carrier contains about 4% dextrose. In some embodiments the pharmaceutically acceptable carrier contains about 5% dextrose. In some embodiments the pharmaceutically acceptable carrier contains about 6% dextrose. In some embodiments the pharmaceutically acceptable carrier contains about 7% dextrose. In some embodiments the pharmaceutically acceptable carrier contains about 8% dextrose. In some embodiments the pharmaceutically acceptable carrier contains about 9% dextrose. In some embodiments the pharmaceutically acceptable carrier contains about 10% dextrose. In some embodiments of the described method includes the use of a composition including a DNA plasmid encoding SDF-1 and a pharmaceutically acceptable carrier containing about 2% dextrose. In some embodiments of the described method includes the use of a composition including a DNA plasmid encoding SDF-1 having the sequence of SEQ ID NO: 1 and a pharmaceutically acceptable carrier containing about 2% dextrose. In some embodiments of the described method includes the use of a composition including a DNA plasmid encoding SDF-1 and a pharmaceutically acceptable carrier containing about 3% dextrose. In some embodiments of the described method includes the use of a composition including a DNA plasmid encoding SDF-1 having the sequence of SEQ ID NO: 1 and a pharmaceutically acceptable carrier containing about 3% dextrose. In some embodiments of the described method includes the use of a composition including a DNA plasmid encoding SDF-1 and a pharmaceutically acceptable carrier containing about 4% dextrose. In some embodiments of the described method includes the use of a composition including a DNA plasmid encoding SDF-1 having the sequence of SEQ ID NO: 1 and a pharmaceutically acceptable carrier containing about 4% dextrose. In some embodiments of the described method includes the use of a composition including a DNA plasmid encoding SDF-1 and a pharmaceutically acceptable carrier containing about 5% dextrose. In some embodiments of the described method includes the use of a composition including a DNA plasmid encoding SDF-1 having the sequence of SEQ ID NO: 1 and a pharmaceutically acceptable carrier containing about 5% dextrose. In some embodiments of the described method includes the use of a composition including a DNA plasmid encoding SDF-1 and a pharmaceutically acceptable carrier containing about 6% dextrose. In some embodiments of the described method includes the use of a composition including a DNA plasmid encoding SDF-1 having the sequence of SEQ ID NO: 1 and a pharmaceutically acceptable carrier containing about 6% dextrose. In some embodiments of the described method includes the use of a composition including a DNA plasmid encoding SDF-1 and a pharmaceutically acceptable carrier containing about 7% dextrose. In some embodiments of the described method includes the use of a composition including a DNA plasmid encoding SDF-1 having the sequence of SEQ ID NO: 1 and a pharmaceutically acceptable carrier containing about 7% dextrose. In some embodiments of the described method includes the use of a composition including a DNA plasmid encoding SDF-1 and a pharmaceutically acceptable carrier containing about 8% dextrose. In some embodiments of the described method includes the use of a composition including a DNA plasmid encoding SDF-1 having the sequence of SEQ ID NO: 1 and a pharmaceutically acceptable carrier containing about 8% dextrose.

In addition to a pharmaceutically acceptable carrier, the compositions described herein that contain a polynucleotide encoding SDF-1 for use in the described methods of treating cardiomyopathy, ischemic cardiomyopathy, or non-ischemic cardiomyopathy can further include a buffer. In some embodiments the described compositions may include a buffer containing dibasic acid, carbonic acid and polybasic acid, phosphoric acid or suitable salts thereof. In some embodiments the described compositions can incorporate a buffer to maintain a physiologic pH. In some embodiments the described compositions can incorporate a buffer to maintain a slightly acid pH. In some embodiments the described compositions can incorporate a buffer to maintain a slightly basic pH. In some embodiments the described compositions can incorporate a buffer to promote a pH of about 5.4 to about 7.4.

The compositions described herein that contain a polynucleotide encoding SDF-1 for use in the described methods of treating cardiomyopathy, ischemic cardiomyopathy, or non-ischemic cardiomyopathy can include a polynucleotide encoding SDF-1 at a concentration of about 0.125 mg/ml to about 2.0 mg/ml. In some embodiments the described compositions have about 0.125 mg/ml of a polynucleotide encoding SDF-1. In some embodiments the described compositions have about 0.25 mg/ml of a polynucleotide encoding SDF-1. In some embodiments the described compositions have about 0.375 mg/ml of a polynucleotide encoding SDF-1. In some embodiments the described compositions have about 0.5 mg/ml of a polynucleotide encoding SDF-1. In some embodiments the described compositions have about 0.625 mg/ml of a polynucleotide encoding SDF-1.

In some embodiments the described compositions have about 0.75 mg/ml of a polynucleotide encoding SDF-1. In some embodiments the described compositions have about 0.875 mg/ml of a polynucleotide encoding SDF-1. In some embodiments the described compositions have about 1 mg/ml of a polynucleotide encoding SDF-1. In some embodiments the described compositions have about 1.125 mg/ml of a polynucleotide encoding SDF-1. In some embodiments the described compositions have about 1.25 mg/ml of a polynucleotide encoding SDF-1. In some embodiments the described compositions have about 1.375 mg/ml of a polynucleotide encoding SDF-1. In some embodiments the described compositions have about 1.5 mg/ml of a polynucleotide encoding SDF-1. In some embodiments the described compositions have about 1.625 mg/ml of a polynucleotide encoding SDF-1. In some embodiments the described compositions have about 1.75 mg/ml of a polynucleotide encoding SDF-1. In some embodiments the described compositions have about 1.875 mg/ml of a polynucleotide encoding SDF-1. In some embodiments the described compositions have about 2 mg/ml of a polynucleotide encoding SDF-1. In each of the described embodiments the polynucleotide encoding SDF-1 can be a DNA plasmid having the sequence of SEQ ID NO:1.

The described compositions containing a polynucleotide encoding SDF-1 for use in the described methods of treating cardiomyopathy, ischemic cardiomyopathy, or non-ischemic cardiomyopathy can be used to administer a total amount of from about 10 mg to about 75 mg of the polynucleotide to the subject. In some embodiments the total amount of the polynucleotide encoding SDF-1 delivered to the subject is about 10 mg. In some embodiments the total amount of the polynucleotide encoding SDF-1 delivered to the subject is about 15 mg. In some embodiments the total amount of the polynucleotide encoding SDF-1 delivered to the subject is about 20 mg. In some embodiments the total amount of the polynucleotide encoding SDF-1 delivered to the subject is about 25 mg. In some embodiments the total amount of the polynucleotide encoding SDF-1 delivered to the subject is about 30 mg. In some embodiments the total amount of the polynucleotide encoding SDF-1 delivered to the subject is about 35 mg. In some embodiments the total amount of the polynucleotide encoding SDF-1 delivered to the subject is about 40 mg. In some embodiments the total amount of the polynucleotide encoding SDF-1 delivered to the subject is about 45 mg. In some embodiments the total amount of the polynucleotide encoding SDF-1 delivered to the subject is about 50 mg. In some embodiments the total amount of the polynucleotide encoding SDF-1 delivered to the subject is about 55 mg. In some embodiments the total amount of the polynucleotide encoding SDF-1 delivered to the subject is about 60 mg. In some embodiments the total amount of the polynucleotide encoding SDF-1 delivered to the subject is about 65 mg. In some embodiments the total amount of the polynucleotide encoding SDF-1 delivered to the subject is about 70 mg. In some embodiments the total amount of the polynucleotide encoding SDF-1 delivered to the subject is about 75 mg. In each of the described embodiments the polynucleotide encoding SDF-1 can be a DNA plasmid having the sequence of SEQ ID NO:1.

The described compositions containing a polynucleotide encoding SDF-1 for use in the described methods of treating cardiomyopathy, ischemic cardiomyopathy, or non-ischemic cardiomyopathy can be delivered to the subject's heart in a total volume of about 30 ml to about 100 ml. In one embodiment the total volume of the composition containing a polynucleotide encoding SDF-1 that is delivered to the heart of a subject is about 30 ml. In one embodiment the total volume of the composition containing a polynucleotide encoding SDF-1 that is delivered to the heart of a subject is about 35 ml. In one embodiment the total volume of the composition containing a polynucleotide encoding SDF-1 that is delivered to the heart of a subject is about 40 ml. In one embodiment the total volume of the composition containing a polynucleotide encoding SDF-1 that is delivered to the heart of a subject is about 45 ml. In one embodiment the total volume of the composition containing a polynucleotide encoding SDF-1 that is delivered to the heart of a subject is about 50 ml. In one embodiment the total volume of the composition containing a polynucleotide encoding SDF-1 that is delivered to the heart of a subject is about 55 ml. In one embodiment the total volume of the composition containing a polynucleotide encoding SDF-1 that is delivered to the heart of a subject is about 60 ml. In one embodiment the total volume of the composition containing a polynucleotide encoding SDF-1 that is delivered to the heart of a subject is about 65 ml. In one embodiment the total volume of the composition containing a polynucleotide encoding SDF-1 that is delivered to the heart of a subject is about 70 ml. In one embodiment the total volume of the composition containing a polynucleotide encoding SDF-1 that is delivered to the heart of a subject is about 75 ml. In one embodiment the total volume of the composition containing a polynucleotide encoding SDF-1 that is delivered to the heart of a subject is about 80 ml. In one embodiment the total volume of the composition containing a polynucleotide encoding SDF-1 that is delivered to the heart of a subject is about 85 ml. In one embodiment the total volume of the composition containing a polynucleotide encoding SDF-1 that is delivered to the heart of a subject is about 90 ml. In one embodiment the total volume of the composition containing a polynucleotide encoding SDF-1 that is delivered to the heart of a subject is about 95 ml. In one embodiment the total volume of the composition containing a polynucleotide encoding SDF-1 that is delivered to the heart of a subject is about 100 ml. In each of the described embodiments the polynucleotide encoding SDF-1 can be a DNA plasmid having the sequence of SEQ ID NO:1.

The described methods of treating a subject with an cardiomyopathy, ischemic cardiomyopathy, or non-ischemic cardiomyopathy by administering to the subject's heart, via percutaneous retrograde coronary sinus perfusion, a polynucleotide encoding SDF-1 can be carried out in a variety of ways, some of which vary by the location of where the balloon catheter is inflated in the subject. In one embodiment, the method may be carried out by inserting a catheter with a balloon into the femoral vein, internal jugular vein, subclavian vein, antecubital vein, brachial vein, or a radial vein of the subject, advancing said catheter into the coronary sinus, and inflating the balloon in the coronary sinus of the subject prior to administration of the composition containing the plasmid encoding SDF-1. In one embodiment, the method may be carried out by inserting a catheter with a balloon into the femoral vein, internal jugular vein, subclavian vein, antecubital vein, brachial vein, or a radial vein of the subject, advancing said catheter into the coronary sinus, and inflating the balloon in the middle cardiac vein of the subject prior to administration of the composition containing the plasmid encoding SDF-1. In one embodiment, the method may be carried out by inserting a catheter with a balloon into the femoral vein, internal jugular vein, subclavian vein, antecubital vein, brachial vein, or a radial vein of the subject, advancing said catheter into the coronary sinus, and inflating the balloon in the lesser (small) cardiac vein of the subject prior to administration of the composition containing the plasmid encoding SDF-1. In one embodiment, the method may be carried out by inserting a catheter with a balloon into the femoral vein, internal jugular vein, subclavian vein, antecubital vein, brachial vein, or a radial vein of the subject, advancing said catheter into the coronary sinus, and inflating the balloon in the great cardiac vein of the subject prior to administration of the composition containing the plasmid encoding SDF-1. In some embodiments of the described method a contrast agent may be administered prior to delivery of the composition containing an SDF-1 plasmid to allow for fluoroscopic visualization. In each of the described embodiments the polynucleotide encoding SDF-1 can be a DNA plasmid having the sequence of SEQ ID NO:1. It should be understood that the described methods may be carried out using a variety of different catheter types known to those skilled in the art. For example, suitable catheters may have a compliant balloon or a non-compliant balloon, may be filled with gas or liquid to inflate the balloon, or have other properties varied from the particular catheters described herein.

In one embodiment, the method may be carried out by inserting a catheter with a balloon into the femoral vein, internal jugular vein, subclavian vein, antecubital vein, brachial vein, or a radial vein of the subject, advancing said catheter into the coronary sinus, and inflating the balloon to a pressure of no more than 2 ATM in the coronary sinus of the subject prior to administration of the composition containing the plasmid encoding SDF-1. In one embodiment, the method may be carried out by inserting a catheter with a balloon into the femoral vein, internal jugular vein, subclavian vein, antecubital vein, brachial vein, or a radial vein of the subject, advancing said catheter into the coronary sinus, and inflating the balloon to a pressure of no more than 2 ATM in the middle cardiac vein of the subject prior to administration of the composition containing the plasmid encoding SDF-1. In one embodiment, the method may be carried out by inserting a catheter with a balloon into the femoral vein, internal jugular vein, subclavian vein, antecubital vein, brachial vein, or a radial vein of the subject, advancing said catheter into the coronary sinus, and inflating the balloon to a pressure of no more than 2 ATM in the lesser (small) cardiac vein of the subject prior to administration of the composition containing the plasmid encoding SDF-1. In one embodiment, the method may be carried out by inserting a catheter with a balloon into the femoral vein, internal jugular vein, subclavian vein, antecubital vein, brachial vein, or a radial vein of the subject, advancing said catheter into the coronary sinus, and inflating the balloon to a pressure of no more than 2 ATM in the great cardiac vein of the subject prior to administration of the composition containing the plasmid encoding SDF-1. In one embodiment, the method may be carried out by inserting a catheter with a balloon into the femoral vein, internal jugular vein, subclavian vein, antecubital vein, brachial vein, or a radial vein of the subject, advancing said catheter into the coronary sinus, and inflating the balloon to a pressure of no more than 1 ATM in the coronary sinus of the subject prior to administration of the composition containing the plasmid encoding SDF-1. In one embodiment, the method may be carried out by inserting a catheter with a balloon into the femoral vein, internal jugular vein, subclavian vein, antecubital vein, brachial vein, or a radial vein of the subject, advancing said catheter into the coronary sinus, and inflating the balloon to a pressure of no more than 1 ATM in the middle cardiac vein of the subject prior to administration of the composition containing the plasmid encoding SDF-1. In one embodiment, the method may be carried out by inserting a catheter with a balloon into the femoral vein, internal jugular vein, subclavian vein, antecubital vein, brachial vein, or a radial vein of the subject, advancing said catheter into the coronary sinus, and inflating the balloon to a pressure of no more than 1 ATM in the lesser (small) cardiac vein of the subject prior to administration of the composition containing the plasmid encoding SDF-1. In one embodiment, the method may be carried out by inserting a catheter with a balloon into the femoral vein, internal jugular vein, subclavian vein, antecubital vein, brachial vein, or a radial vein of the subject, advancing said catheter into the coronary sinus, and inflating the balloon to a pressure of no more than 1 ATM in the great cardiac vein of the subject prior to administration of the composition containing the plasmid encoding SDF-1. In some embodiments of the described method a contrast agent may be administered prior to delivery of the composition containing an SDF-1 plasmid to allow for fluoroscopic visualization. In each of the described embodiments the polynucleotide encoding SDF-1 can be a DNA plasmid having the sequence of SEQ ID NO:1.

Once the balloon is inflated, the pharmaceutical composition can be infused through the catheter into the subject over a period of about 2 minutes. In some embodiments of the method, following infusion of the pharmaceutical composition the balloon can remain inflated from about 5 to 15 minutes. In some embodiments of the method, following infusion of the pharmaceutical composition the balloon can remain inflated from about 7 to 12 minutes. In some embodiments of the method, following infusion of the pharmaceutical composition the balloon can remain inflated for about 10 minutes.

Following administration of the described pharmaceutical compositions having a plasmid encoding SDF-1, the SDF-1 protein can be expressed in cells of the heart. In some embodiments the duration of the plasmid in some cells of the heart can be about 10 days. In some embodiments the duration of the plasmid in some cells of the heart can be about 20 days. In some embodiments the duration of the plasmid in some cells of the heart can be about 30 days. In some embodiments the duration of the plasmid in some cells of the heart can be about 40 days. In some embodiments the duration of the plasmid in some cells of the heart can be about 50 days. In some embodiments the duration of the plasmid in some cells of the heart can be about 60 days.

The following examples are provided to describe certain embodiments of the subject matter described herein with greater detail. They are intended to illustrate, not to limit, the embodiments.

### EXAMPLE I

### Retrograde Infusion of Luciferase in Pigs

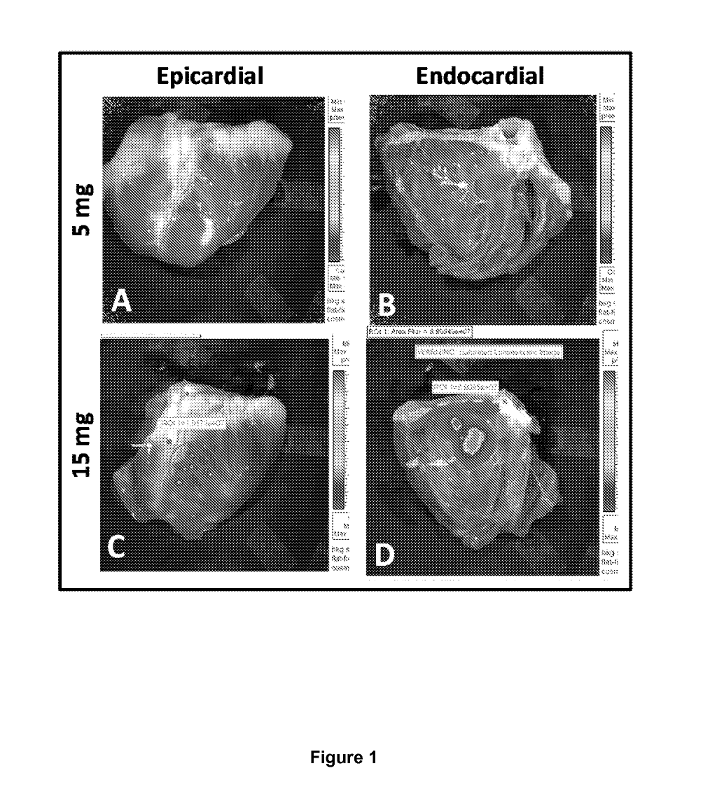

The goal of this study was to determine presence and distribution of protein expression following retrograde delivery of plasmid into previously infarcted myocardium. To achieve this objective, we evaluated luciferase plasmid DNA expression in porcine hearts 3 days after retrograde infusion in pigs 4 months after acute myocardial infarction. The plasmid used in this study (ACL-01110L) has the identical backbone as the plasmid contained in JVS-100 but expresses a luciferase reporter gene instead of SDF-1 to monitor expression. Three adult Yorkshire pigs, after having received an acute myocardial infarction 4 months previously, were infused via retrograde delivery with 40 mls of ACL-01110L containing either 5 mg (1 animal) or 15 mg (2 animals) of plasmid. Retrograde delivery consisted of insertion of a balloon catheter (ARROW Double Lumen Wedge Balloon Catheter) through the coronary sinus into a large coronary vein, inflating the balloon, and infusing ACL-01110L into the coronary venous system. Three days post-infusion, animals were sacrificed and the heart muscle excised. Reporter (luciferase) gene expression was measured in the intact excised heart using a Xenogen imaging system. No expression was detected in the animal receiving the 5 mg dose (FIG. 1(A and B)); whereas, significant protein expression was detected in both animals receiving 15 mg (FIG. 1(C and D)). These results suggest there is a minimum dose required to elicit JVS-100 protein expression via retrograde infusion that is between 5-15 mg.

### EXAMPLE II

### Plasmid Expression after Retrograde Coronary Sinus Delivery

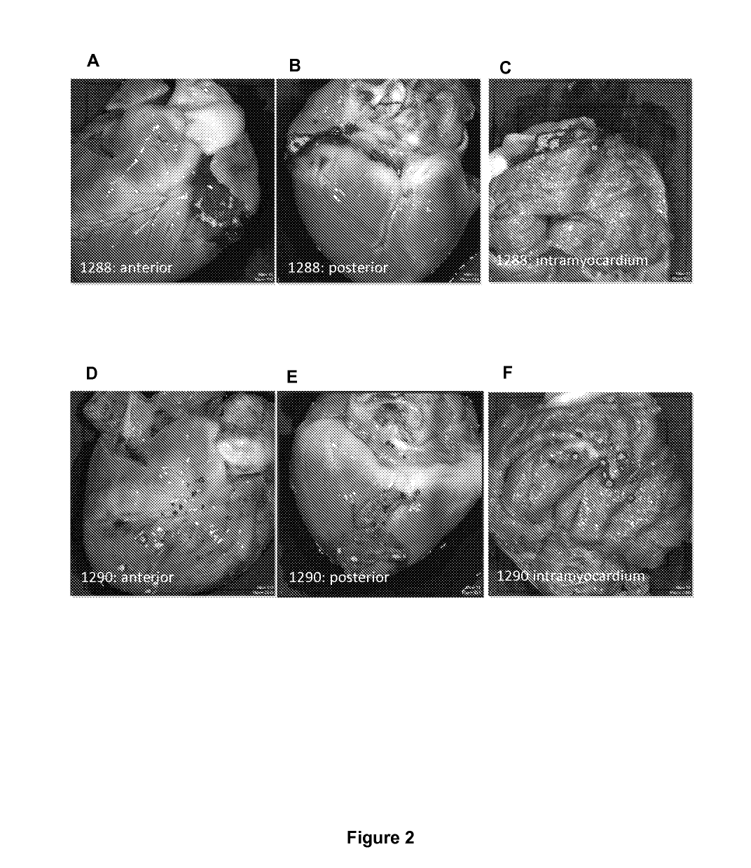

To determine impact of infusion site plasmid biodistribution was tested in 4 pigs using either the Cook Advance 35LP catheter (n=2) or using a Cook Advance® 35LP catheter in combination with a Cook Cantata® microcatheter. Four (4/4) domestic swine were anesthetized and vascular access was obtained via the right jugular vein. After measurements were taken of the great cardiac vein, an appropriately sized Cook Advance® 35LP balloon catheter was placed into the cardiac vein. In two of the four animals, 40 mls of luciferase tagged JVS-100, ACL-01110L (1 mg/ml), was infused over the course of 2 minutes. Ten (10) mls of plasmid was infused through the microcatheter at each of the four locations. Following infusion, occlusion was maintained for 10 minute. Forty-eight (48) hours after infusion procedures, animals were sacrifices and the hearts were excised. Each heart was placed in D-Luciferase substrate then imaged for luciferase expression (FIG. 2(A-F)).

The results from this study showed robust protein expression in the heart tissue of both animals following retrograde infusion through the Cook Advance 35 LP alone. Based on these results, we conclude that retrograde coronary sinus administration of the plasmid through the Cook Advance 35LP balloon catheter resulted in distribution to the heart, which remained detectable after 48 hours.

### EXAMPLE III

### Retrograde Coronary Sinus Delivery of JVS-100 in a Porcine Pig Model

Delivery of JVS-100 was tested using retrograde coronary sinus administration in an established porcine occlusion/reperfusion model of MI. In this study, 24 pigs were injected 30 days following occlusion of the left anterior descending coronary artery (LAD) with a single dose of JVS-100 at one of two dose levels, i.e., 15 mg, or 45 mg (expressed as total DNA delivered from all injections). The pigs all had systolic dysfunction (LVEF<40%) and evidence of cardiac remodeling (LVESV>55 ml) at the time of injection (30 days following MI). Four animals in each group were sacrificed at either 3 or 60 days. Animals were assessed for safety endpoints including biodistribution of the plasmid to the heart and other tissues, clinical pathology (hematology, serum chemistries, coagulation) and histopathological examination of tissues at day 3, and day 60. Measurements of efficacy were performed on animals at 60 days.

Administration of the test article at doses of 15 and 45 mg in 40 ml of a 5% dextrose solution via retrograde perfusion was not associated with any adverse effects. Only one pig died prior to scheduled necropsy. This death was due to complication of the cardiac injury in the animal model and was not related to test article administration. Administration of the test article was not associated with any mortality, clinical findings, changes in body weight, changes in clinical pathology endpoints, or adverse macroscopic findings. There were no test related effects among hematology parameters in any treatment group relative to vehicle controls. Administration of the test article was associated with a transient, slight increase at 6 hours post infusion in creatinine kinase MB and Troponin-I relative to controls. The magnitude of this increase was small in comparison to the clinically relevant increases noted following MI.

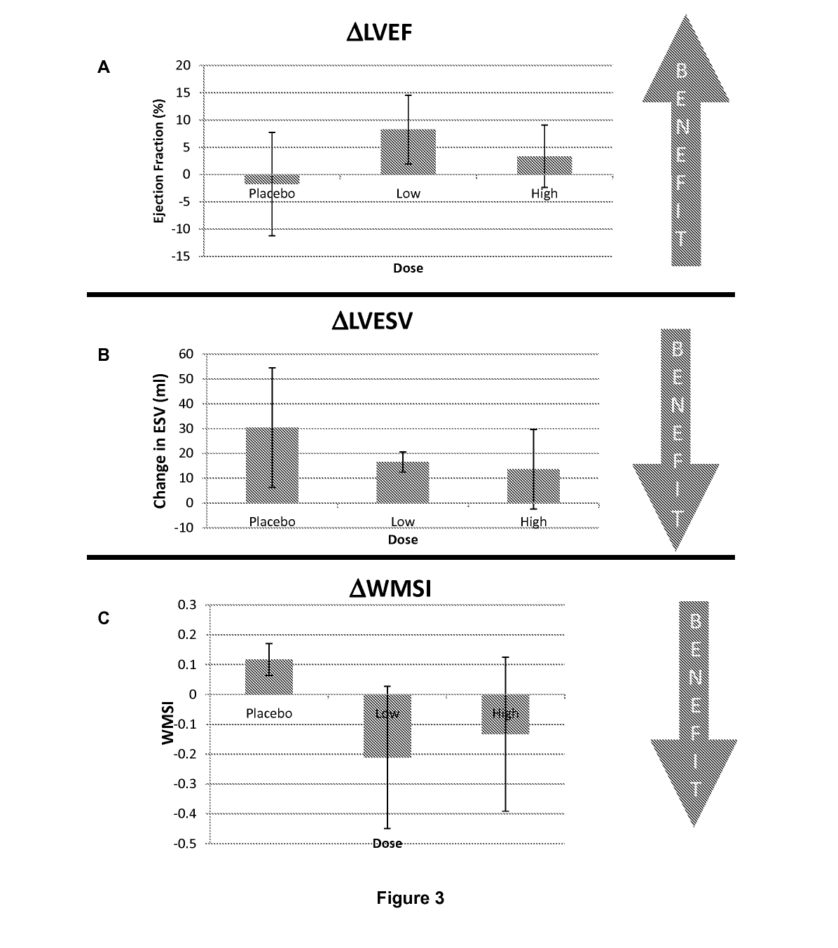

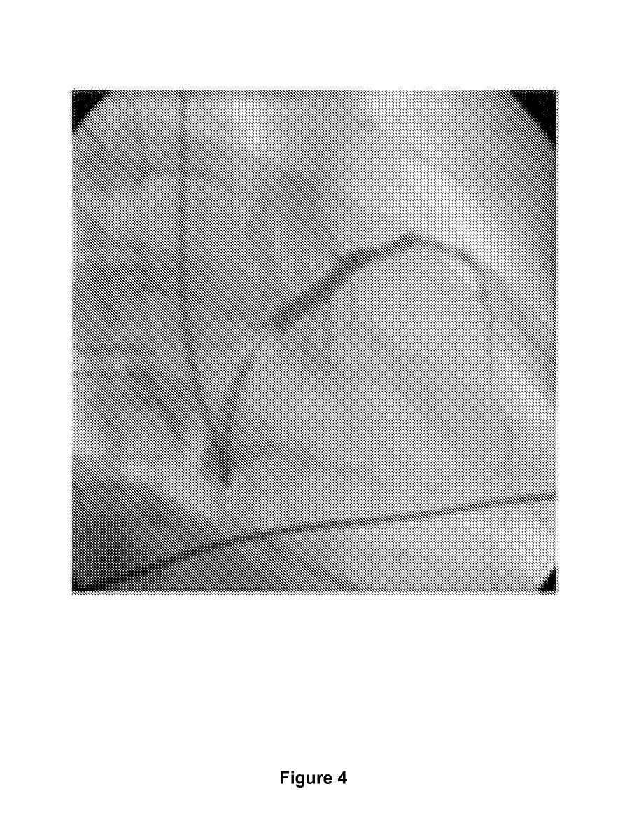

Efficacy was measured by monitoring cardiac size and function by serial echocardiography at prior to dosing and at Day 60. Control animals show a trend toward increased (i.e. worsening) LVESV and no improvement in function (i.e. LVEF and wall motion score index (WMSI)) at 60 days, consistent with this heart failure model. In contrast, both the low and high dose groups demonstrated a trend towards improved cardiac function (LVEF and WMSI) and attenuation of LV remodeling (LVESV) (FIG. 4). Although there was no statistically significant treatment effect on LVEF or LVESV, there is a statistically significant improvement in WMSI in the low dose treated group compared to the placebo control (p=0.029) (FIG. 3(C)). No significant difference in baseline values was observed for any of the parameters using these analyses between control and treated groups. Baseline is defined as just prior to treatment at day 0, thirty days after the infarction. There was no significant change from baseline to day 60 in any of the parameters examined in either the treated or control groups.

When compared to each other, there was no statistical difference measured between the dose groups. Additionally, no non-statistical trend emerged as to which dose was most effective, with functional measurements demonstrating slightly more benefit in the low dose (LVEF and WMSI) while LV remodeling measurements showed slightly less worsening in the high dose treatment group (LVESV and LVEDV).

JVS-100 distribution in cardiac and non-cardiac tissues was measured 3 and 60 days after injection. In cardiac tissue, at each time point, JVS-100 plasmid was present in all sections of heart analyzed, demonstrating extensive distribution following instillation into the coronary sinus. Low levels of JVS-100 were detected in non-target organs at day 3, with the highest levels noted in the kidney. By approximately 60 days after treatment, JVS-100 was completely cleared from all of the non-target organs examined Limited amounts of JVS-100 were only detected in some right and left ventricles of the swine heart. In the case of treatment at 15 mg in 40 mL, one distal coronary sinus tissue was also detected with JVS-100 at 98 copies per μg of swine tissue DNA.

The results of this porcine MI efficacy and safety study demonstrate that JVS-100 administered via retrograde coronary sinus delivery at doses of 15 mg and 30 mg showed evidence of therapeutic benefit. No evidence of toxicological effects was observed at either dose.

### EXAMPLE IV

### Retrograde Infusion of JVS-100 in Human Subjects

We plan to deliver JVS-100 via retrograde infusion to subjects with ischemic heart failure using a balloon occlusion catheter system. Using aseptic technique and local anesthesia, an access sheath will be inserted into the most distal portion of the femoral vein. Access to the coronary sinus is achieved using standard catheter techniques. Once access to the coronary sinus has been achieved, the balloon occlusion catheter will be placed into the coronary sinus using standard catheter techniques. Following gentle advancement of the catheter into the coronary sinus, the balloon will be placed in a non-obstructing mid-position. The balloon should be positioned in the coronary sinus in one of the following positions near the infarcted area as determined by the treating physician's clinical judgment:

1) Coronary Sinus,

2) Great Cardiac Vein,

3) Anterior Interventricular Vein, and

4) Posterolateral Vein.

The particular characteristics of each case may require placement of the balloon in a position other than these four positions (FIG. 4). The actual placement will be recorded to determine the frequency of this occurrence. Once in place, the balloon will be inflated to no more than 2 ATM and the total volume of 40 ml JVS-100 will be divided into four 10 mL syringes and infused through the catheter lumen into the coronary sinus for a total of 2 minutes. The balloon will remain inflated for 10 minutes after infusion to permit the diffusion of the plasmid into the cardiac tissue.

The proposed doses of JVS-100, 15, 30, and 45 mg, to be delivered by retrograde infusion are based on accumulated data, which are summarized in Table 1. The 15 mg (0.375 mg/ml) dose is equivalent to the lowest dose which demonstrated protein expression of JVS-100 and was shown to be efficacious. The 30 mg (0.75 mg/ml) dose is greater than the lowest dose that demonstrated protein expression of JVS-100 in our JVS-100 Retrograde Infusion Expression study efficacy in the GLP study, JVS-100 delivery into a porcine model of heart failure (1564-003), when delivered by endomyocardial injection. The 45 mg (1.125 mg/ml) dose demonstrated efficacy in a GLP retrograde study and is less than half of the 100 mg dose at which no toxicity was observed in an exploratory study.

TABLE 1
Support for Proposed Doses of JVS-100 Administered by Retrograde Infusion

| | N/Dose | Product | Results |
|---|---|---|---|
| Porcine Retrograde | N = 3 | ACL-01110L | Significant expression at 15 mg dose |
| Infusion Study | 5 mg (n = 1) | (JVS-100 expresses | No luciferase expression at 5 mg dose |
| | 15 mg (n = 2) | luciferase gene instead | |
| | | of SDF-1) | |
| Porcine Retrograde | N = 4 | ACL-01110L | Significant expression at 40 mg with Cook |
| Infusion Study | 40 mg (n = 4) | (JVS-100 expresses | Advance 35LP catheter alone. |
| | | luciferase gene instead | Low expression with the addition of |
| | | of SDF-1) | microcatheter (n = 2) |
| Porcine MI | N = 24 | JVS-100 | JVS-100 safe at all doses tested (15 and 45 mg) |
| Retrograde Coronary | Control (n = 8) | | 15 and 30 mgs show a trend toward efficacy |
| Sinus Safety and | 15 mg (n = 8) | | as measured by % change in LVESV and |
| Efficacy Study | 45 mg (n = 8) | | LVEF, and WMSI with a significant |
| | | | improvement in WMSI in the 15 mg dose. |

SEQ ID NO: 1
AGATCTCCTAGGGAGTCCGTTACATAACTTACGGTAAATGGCCCGCCTGGCTGACCG
CCCAACGACCCCCGCCCATTGACGTCAATAATGACGTATGTTCCCATAGTAACGCCA
ATAGGGACTTTCCATTGACGTCAATGGGTGGAGTATTTACGGTAAACTGCCCACTTG
GCAGTACATCAAGTGTATCATATGCCAAGTACGCCCCCTATTGACGTCAATGACGGT
AAATGGCCCGCCTGGCATTATGCCCAGTACATGACCTTATGGGACTTTCCTACTTGG
CAGTACATCTACGTATTAGTCATCGCTATTACCATGGTGATGCGGTTTTGGCAGTAC
ATCAATGGGCGTGGATAGCGGTTTGACTCACGGGGATTTCCAAGTCTCCACCCCATT
GACGTCAATGGGAGTTTGTTTTGGCACCAAAATCAACGGGACTTTCCAAAATGTCGT
AACAACTCCGCCCCATTGACGCAAATGGGCGGTAGGCGTGTACGGTGGGAGGTCTA
TATAAGCAGAGCTCGTTTAGTGAACCGTCAGATCGCCTGGAGACGCCATCCACGCTG
TTTTGACCTCCATAGAAGACACCGGGACCGATCCAGCCTCCGCGGCCGGGAACGGT
GCATTGGAACGCGGATTCCCCGTGCCAAGAGTGACGTAAGTACCGCCTATAGAGTCT
ATAGGCCCACCCCCTTGGCTTCTTATGCATGCTATACTGTTTTTGGCTTGGGGTCTAT
ACACCCCCGCTTCCTCATGTTATAGGTGATGGTATAGCTTAGCCTATAGGTGTGGGT
TATTGACCATTATTGACCACTCCCCTATTGGTGACGATACTTTCCATTACTAATCCAT
AACATGGCTCTTTGCCACAACTCTCTTTATTGGCTATATGCCAATACACTGTCCTTCA
GAGACTGACACGGACTCTGTATTTTTACAGGATGGGGTCTCATTTATTATTTACAAA
TTCACATATACAACACCACCGTCCCCAGTGCCCGCAGTTTTTATTAAACATAACGTG
GGATCTCCACGCGAATCTCGGGTACGTGTTCCGGACATGGGCTCTTCTCCGGTAGCG
GCGGAGCTTCTACATCCGAGCCCTGCTCCCATGCCTCCAGCGACTCATGGTCGCTCG
GCAGCTCCTTGCTCCTAACAGTGGAGGCCAGACTTAGGCACAGCACGATGCCCACC
ACCACCAGTGTGCCGCACAAGGCCGTGGCGGTAGGGTATGTGTCTGAAAATGAGCT
CGGGGAGCGGGCTTGCACCGCTGACGCATTTGGAAGACTTAAGGCAGCGGCAGAAG
AAGATGCAGGCAGCTGAGTTGTTGTGTTCTGATAAGAGTCAGAGGTAACTCCCGTTG
CGGTGCTGTTAACGGTGGAGGGCAGTGTAGTCTGAGCAGTACTCGTTGCTGCCGCGC
GCGCCACCAGACATAATAGCTGACAGACTAACAGACTGTTCCTTTCCATGGGTCTTT
TCTGCAGTCACCGTCCTTGCCATCGGTGACCACTAGTGGCTCGCATCTCTCCTTCACG
CGCCCGCCGCCCTACCTGAGGCCGCCATCCACGCCGGTTGAGTCGCGTTCTGCCGCC
TCCCGCCTGTGGTGCCTCCTGAACTGCGTCCGCCGTCTAGGTAAGTTTAAAGCTCAG
GTCGAGACCGGGCCTTTGTCCGGCGCTCCCTTGGAGCCTACCTAGACTCAGCCGGCT
CTCCACGCTTTGCCTGACCCTGCTTGCTCAACTCTACGTCTTTGTTTCGTTTTCTGTTC
TGCGCCGTTACAGATCGGTACCAAGCTTGCCACCACCATGAACGCCAAGGTCGTGGT
CGTGCTGGTCCTCGTGCTGACCGCGCTCTGCCTCAGCGACGGGAAGCCCGTCAGCCT
GAGCTACAGATGCCCATGCCGATTCTTCGAAAGCCATGTTGCCAGAGCCAACGTCA
AGCATCTCAAAATTCTCAACACCCCAAACTGTGCCCTTCAGATTGTAGCCCGGCTGA
AGAACAACAACAGACAAGTGTGCATTGACCCGAAGCTAAAGTGGATTCAGGAGTAC
CTGGAGAAAGCCTTAAACAAGTAATCTAGAGGGCCCTATTCTATAGTGTCACCTAAA
TGCTAGAGCTCGCTGATCAGCCTCGACTGTGCCTTCTAGTTGCCAGCCATCTGTTGTT
TGCCCCTCCCCCGTGCCTTCCTTGACCCTGGAAGGTGCCACTCCCACTGTCCTTTCCT
AATAAAATGAGGAAATTGCATCGCATTGTCTGAGTAGGTGTCATTCTATTCTGGGGG
GTGGGGTGGGGCAGGACAGCAAGGGGGAGGATTGGGAAGACAATAGCAGGCATGC
TGGGGATGCGGTGGGCTCTATGGCTTCTGAGGCGGAAAGAACCAGGGCCGCGGTGG
CCATCATGACCAAAATCCCTTAACGTGAGTTTTCGTTCCACTGAGCGTCAGACCCCG
TAGAAAAGATCAAAGGATCTTCTTGAGATCCTTTTTTTCTGCGCGTAATCTGCTGCTT
GCAAACAAAAAAACCACCGCTACCAGCGGTGGTTTGTTTGCCGGATCAAGAGCTAC
CAACTCTTTTTCCGAAGGTAACTGGCTTCAGCAGAGCGCAGATACCAAATACTGTTC
TTCTAGTGTAGCCGTAGTTAGGCCACCACTTCAAGAACTCTGTAGCACCGCCTACAT
ACCTCGCTCTGCTAATCCTGTTACCAGTGGCTGCTGCCAGTGGCGATAAGTCGTGTC
TTACCGGGTTGGACTCAAGACGATAGTTACCGGATAAGGCGCAGCGGTCGGGCTGA
ACGGGGGGTTCGTGCACACAGCCCAGCTTGGAGCGAACGACCTACACCGAACTGAG
ATACCTACAGCGTGAGCTATGAGAAAGCGCCACGCTTCCCGAAGGGAGAAAGGCGG
ACAGGTATCCGGTAAGCGGCAGGGTCGGAACAGGAGAGCGCACGAGGGAGCTTCC
AGGGGGAAACGCCTGGTATCTTTATAGTCCTGTCGGGTTTCGCCACCTCTGACTTGA
GCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGAATTCAGA
AGAACTCGTCAAGAAGGCGATAGAAGGCGATGCGCTGCGAATCGGGAGCGGCGAT
ACCGTAAAGCACGAGGAAGCGGTCAGCCCATTCGCCGCCAAGCTCTTCAGCAATAT
CACGGGTAGCCAACGCTATGTCCTGATAGCGGTCCGCCACACCCAGCCGGCCACAG
TCGATGAATCCAGAAAAGCGGCCATTTTCCACCATGATATTCGGCAAGCAGGCATCG
CCATGGGTCACGACGAGATCCTCGCCGTCGGGCATGCTCGCCTTGAGCCTGGCGAAC
AGTTCGGCTGGCGCGAGCCCCTGATGCTCTTCGTCCAGATCATCCTGATCGACAAGA
CCGGCTTCCATCCGAGTACGTGCTCGCTCGATGCGATGTTTCGCTTGGTGGTCGAAT
GGGCAGGTAGCCGGATCAAGCGTATGCAGCCGCCGCATTGCATCAGCCATGATGGA
TACTTTCTCGGCAGGAGCAAGGTGAGATGACAGGAGATCCTGCCCCGGCACTTCGC
CCAATAGCAGCCAGTCCCTTCCCGCTTCAGTGACAACGTCGAGCACAGCTGCGCAA
GGAACGCCCGTCGTGGCCAGCCACGATAGCCGCGCTGCCTCGTCTTGCAGTTCATTC
AGGGCACCGGACAGGTCGGTCTTGACAAAAAGAACCGGGCGCCCCTGCGCTGACAG
CCGGAACACGGCGGCATCAGAGCAGCCGATTGTCTGTTGTGCCCAGTCATAGCCGA
ATAGCCTCTCCACCCAAGCGGCCGGAGAACCTGCGTGCAATCCATCTTGTTCAATCA
TGCGAAACGATCCTCATCCTGTCTCTTGATC

1. A method of treating a subject with an ischemic cardiomyopathy comprising administering to the subject's heart, via percutaneous retrograde coronary sinus perfusion, a pharmaceutical composition that comprises a DNA plasmid encoding SDF-1 and a pharmaceutically acceptable carrier or diluent.

2. The method according to claim 1 wherein said DNA plasmid comprises a polynucleotide having the sequence of SEQ ID NO:1.

3. The method according to claim 1, wherein the subject is suffering from an acute myocardial infarction.

4. The method according to claim 1, wherein the subject has a known history of chronic systolic dysfunction and/or has previously suffered a myocardial infarction.

5. The method according to claim 1 wherein the concentration of said DNA plasmid in said composition is from about 0.125 mg/ml to about 2.0 mg/ml.

6. The method according to claim 5, wherein the concentration of said DNA plasmid in said composition is from about 0.375 mg/ml to about 1.125 mg/ml.

7. The method according to claim 1 wherein the total amount of DNA plasmid administered to said subject of from about 10 mg to about 75 mg.

8. The method according to claim 7 wherein the total amount of DNA plasmid administered to said subject is from about 15 mg to about 45 mg.

9. The method according to claim 8 wherein the total amount of DNA plasmid administered to said subject is about 30 mg.

10. The method according to claim 1 wherein the total volume of said pharmaceutical composition delivered to the subject's heart is from about 30 to about 100 ml.

11. The method according to claim 10, wherein the total volume of said pharmaceutical composition delivered to the subject's heart is about 40 ml.

12. The method according to claim 1 wherein said pharmaceutically acceptable carrier or diluent is 5% dextrose.

13. The method according to claim 12, wherein said pharmaceutically acceptable carrier or diluent further comprises an inert buffer.

14. The method according to claim 1 in which said percutaneous retrograde coronary sinus perfusion administration comprises insertion of a catheter comprising a balloon into the femoral vein of the subject, advancing said catheter into the coronary sinus, and inflating said balloon in the coronary sinus, the middle cardiac vein, the lesser (small) cardiac vein or the great cardiac vein.

15. The method according to claim 14, wherein said balloon is inflated to a pressure of no more than 2 ATM.

16. The method according to claim 14, wherein said pharmaceutical composition is infused through said catheter into the coronary sinus, the middle cardiac vein, the lesser (small) cardiac vein or the great cardiac vein over a period of about 2 minutes, and inflation of said balloon is maintained for a period of about 10 minutes following said infusion.

17.-18. (canceled)


SEQUENCE LISTING (1 sequence)

SEQ ID NO: 1 - DNA, 3948 bp, Artificial Sequence
  Description of Artificial Sequence Synthetic polynucleotide
agatctcctagggagtccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgccatcggtgaccactagtggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatcggtaccaagcttgccaccaccatgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacaccccaaactgtgcccttcagattgtagcccggctgaagaacaacaacagacaagtgtgcattgacccgaagctaaagtggattcaggagtacctggagaaagccttaaacaagtaatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagggccgcggtggccatcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatc